(19) United States
(12) Patent Application Publication
(10) Pub. No.: US 2023/0301778 A1
(43) Pub. Date: Sep. 28, 2023

(54) MULTI-LAYER STRUCTURE FOR AN IMPROVED PRESBYOPIA-CORRECTING LENS AND METHODS OF MANUFACTURING SAME

(71) Applicant: Alcon Inc., Fribourg (CH)

(72) Inventors: Hao Zhang, Keller, TX (US); Wei Lu, Colleyville, TX (US); John Janders, Fort Worth, TX (US); Shinwook Lee, Arlington, TX (US); Sumit Paliwal, Grapevie, TX (US); Xin Hong, Fort Worth, TX (US); Shridhar Natarajan, Arlington, TX (US)

(21) Appl. No.: 18/187,291

(22) Filed: Mar. 21, 2023

Related U.S. Application Data
(60) Provisional application No. 63/364,813, filed on May 17, 2022.
(60) Provisional application No. 63/363,828, filed on Apr. 29, 2022.
(60) Provisional application No. 63/269,719, filed on Mar. 22, 2022.

(51) Int. Cl. A61F 2/16

(52) U.S. Cl. CPC A61F 2/1637; G02B 3/12

(57) ABSTRACT

Certain embodiments provide an intraocular lens (IOL) including a lens body having an anterior lens element and a posterior lens element, and an optical fluid in a cavity formed between the anterior lens element and the posterior lens element. The anterior lens element and the posterior lens element each comprise a lens material having a first Abbe number, and the optical fluid has a second Abbe number that is less than the first Abbe number.

### BACKGROUND

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an intraocular lenses (IOLs).

IOLs are used for cataract surgery to replace the natural lens of the eye and correct refractive errors. Among them are presbyopia-correcting lenses, which include extended depth of focus (EDOF) IOLs. However, the use of such presbyopia-correcting lenses may result in associated visual disturbances such as halos or glare.

### SUMMARY

Aspects of the present disclosure provide an intraocular lens (IOL) including a lens body having an anterior lens element and a posterior lens element, and an optical fluid in a cavity formed between the anterior lens element and the posterior lens element. The anterior lens element and the posterior lens element each comprise a lens material having a first Abbe number, and the optical fluid has a second Abbe number that is less than the first Abbe number.

Aspects of the present disclosure also provide a method for fabricating an intraocular lens (IOL). The method includes fabricating an anterior lens element and a posterior lens element, bonding the anterior lens element and the posterior lens element to form a cavity therebetweeen, and filling the cavity with an optical fluid. The anterior lens element and the posterior lens element each comprise a lens material having a first Abbe number, and the optical fluid has a second Abbe number that is less than the first Abbe number.

Aspects of the present disclosure further provide a method for configuring an intraocular lens (IOL). The method includes computing a radius of curvature of an anterior lens element of a lens body of an IOL and a radius of curvature of a posterior lens element of the lens body of the IOL, based on a lens base power of the IOL, a first refractive index and a first Abbe number of the anterior lens element and the posterior lens element, and a second refractive index and a second Abbe number of an optical fluid to fill a cavity between the anterior lens element and the posterior lens element, and a refractive index and an Abbe number of an optical fluid to fill a cavity between the anterior lens element and the posterior lens element, and forming the IOL or causing the IOL to be formed based on the computed radii of curvature of the anterior lens element and the posterior lens element.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A depicts a top view of an intraocular lens (IOL), according to certain embodiments.

FIG. 1B depicts a side view of a portion of the IOL of FIG. 1A, according to certain embodiments.

FIG. 1C depicts a side view of a portion of the IOL of FIG. 1A, according to certain embodiments.

FIG. 2 depicts example operations for fabricating an IOL, according to certain embodiments.

FIG. 3 depicts an example system for designing, configuring, and/or forming an IOL, according to certain embodiments.

FIG. 4 depicts example operations for forming an IOL, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein provide an intraocular lens (IOL) having a triplet lens body for chromatic aberration correction, and methods and systems for fabricating the same. In certain embodiments, the IOL is an extended depth of focus (EDOF) IOL. In certain embodiments, the IOL having a triplet lens body includes an anterior lens element, a posterior lens element, and an optical fluid filling a cavity formed between the anterior and posterior lens elements. In certain embodiments, a method for fabricating an IOL with a triplet lens body includes producing an anterior lens element and a posterior lens element, bonding the anterior and posterior lens elements to form a cavity therebetween, and filling the cavity with an optical fluid.

### An IOL With Triplet Lens Body for Chromatic Aberration Correction

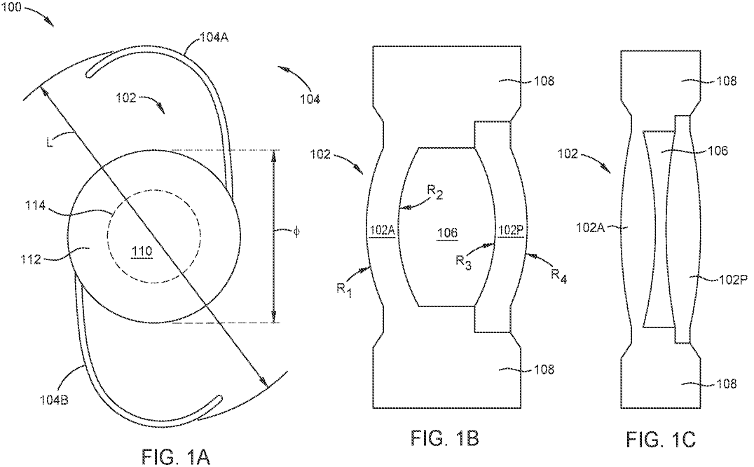

FIG. 1A illustrates a top view of an intraocular lens (IOL) 100, according to certain embodiments. FIGS. 1B and 1C each illustrate a side view of a portion of the IOL 100 with different configurations. The IOL 100 includes a lens body 102 and a haptic portion 104 that is coupled to a peripheral, non-optic portion of the lens body 102.

In the embodiments illustrated in FIGS. 1B and 1C, the lens body 102 has a triplet lens structure, including an anterior lens element 102A and a posterior lens element 102P. The lens elements 102A and 102P are bonded together to form a cavity 106. The cavity 106 is filled with an optical fluid that has a different refractive index and a different Abbe number from those of the lens elements 102A and 102P. The lens elements 102A and 102P have a relatively high refractive index and a relatively low Abbe number, thus leading to relatively large dispersion (i.e., a change in refractive index versus wavelength) and chromatic aberration (i.e., a change in focal point versus wavelength). The optical fluid filling the cavity 106 has a relatively low refractive index and a relatively high Abbe number, thus leading to relatively small dispersion and chromatic aberration. The triplet lens structure, including the lens elements 102A and 102P and the cavity 106 filled with optical fluid, is designed such that the chromatic aberration of the lens elements 102A and 102P is counter-balanced by the chromatic aberration of the optical fluid, by appropriately selecting a radius of curvature R₁ of an external surface of the anterior lens element 102A, a radius of curvature R₂ of an internal surface of the anterior lens element 102A, a radius of curvature R₃ of an internal surface of the posterior lens element 102P, and a radius of curvature R₄ of an external surface of the posterior lens element 102P.

In certain embodiments, as shown in FIG. 1B, the lens body 102 is an achromatic triplet lens. The cavity 106 filled with an optical fluid is a positive (convex) lens. The lens elements 102A and 102P are negative (concave) lenses. An achromatic triplet lens may provide correction to bring two wavelengths of light, e.g., about 0.590 µm (red) and about 0.495 µm (blue), into focus at the same focal point.

In certain embodiments, as shown in FIG. 1C, the lens body 102 is an apochromatic triplet lens. The cavity 106 filled with an optical fluid is a negative (concave) lens. The lens elements 102A and 102P are positive (convex) lenses. An apochromatic triplet lens may provide correction to bring three wavelengths of light, e.g., about 0.620 µm (red), about 0.530 µm (green), and about 0.465 µm (blue), into focus at the same focal point.

The anterior lens element 102A and the posterior lens element 102P may be fabricated of biocompatible material, such as modified poly (methyl methacrylate) (PMMA), modified PMMA hydrogels, hydroxy-ethyl methacrylate (HEMA), PVA hydrogels, other silicone polymeric materials, and hydrophobic acrylic polymeric materials, for example, AcrySof® and Clareon®, available from Alcon, Inc., Fort Worth, Texas. The lens elements 102A and 102P may have a refractive index of between about 1.49 and about 1.56, and an Abbe number of between about 37 and about 53. The optical fluid to fill in the cavity 106 may be an incompressible or substantially incompressible fluid exhibiting a refractive index that is different from the lens elements 102A and 102P. The optical fluid may be silicone oil of ophthalmic grade, such as the optical fluid available from Entegris, Inc., Billerica, Massachusetts. The optical fluid may have a refractive index of between about 1.4 and about 1.55, for example, about 1.43, and an Abbe number of between about 40 and about 50, for example, about 44.4.

In some embodiments, as shown in FIG. 1A, the IOL 100 is an extended depth of focus (EDOF) IOL (with elongated focus) having a lens body 102 with refractive structures on the external surface of the anterior lens element 102A and a smooth convex surface on the external surface of the posterior lens element 102P. As shown in FIG. 1A, the external surface of the anterior lens element 102A may include an inner refractive region 110, an outer annular refractive region 112, and an annular transition region 114 that extends between the inner refractive region 110 and the outer annular refractive region 112. The lens body 102 has a diameter ϕ of between about 4.5 mm and about 7.5 mm, for example, about 6.0 mm.

The IOL 100A further includes a main frame 108 coupled (e.g., glued or welded) to the peripheral portion of the lens body 102 or molded along with a portion of the lens body 102. The main frame 108 includes the haptic portion 104 including radially-extending struts (also referred to as “haptics”) 104A and 104B, and thus the haptics 104A and 104B extend outwardly from the lens body 102 to engage the perimeter wall of the capsular sac of the eye to maintain the lens body 102 in a desired position in the eye. The main frame 108 may be fabricated of biocompatible material, such as modified poly (methyl methacrylate) (PMMA), modified PMMA hydrogels, hydroxy-ethyl methacrylate (HEMA), PVA hydrogels, other silicone polymeric materials, hydrophobic acrylic polymeric materials, for example, AcrySof® and Clareon®, available from Alcon, Inc., Fort Worth, Texas. The haptics 104A and 104B typically have radial-outward ends that define arcuate terminal portions. The terminal portions of the haptics 104A and 104B may be separated by a length L of between about 6 mm and about 22 mm, for example, about 13 mm. The haptics 104A and 104B have a particular length so that the terminal portions create a slight engagement pressure when in contact with the equatorial region of the capsular sac after being implanted. While FIG. 1A illustrates one example configuration of the haptics 104A and 104B, any plate haptics or other types of haptics can be used.

It is noted that the shape and curvatures of the lens body 102 are shown for illustrative purposes only and that other shapes and curvatures are also within the scope of this disclosure. For example, the lens body 102 shown in FIGS. 1B and 1C has a biconvex shape. In other examples, the lens body 102 may have a plano-convex shape, a convexo-concave shape, or a plano-concave shape.

### Fabrication of an IOL With Triplet Lens Body for Chromatic Aberration Correction

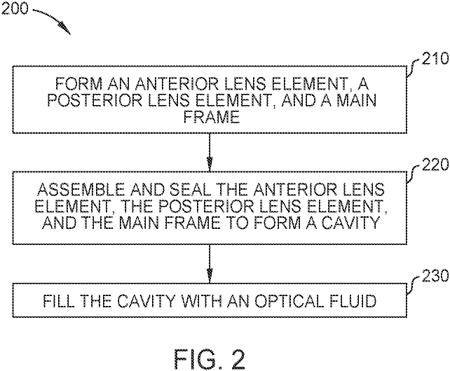

FIG. 2 depicts example operations 200 for forming an IOL with a triplet lens body for chromatic aberration correction, according to certain embodiments.

At step 210, an anterior lens element (e.g., anterior lens element 102A), a posterior lens element (e.g., posterior lens element 102P), and a main frame including haptics (e.g., main frame 108 including haptics 104A and 104B) are formed by a lens manufacturing technique known to one of ordinary skill in the art, such as injection molding and thermal curing processes. The main frame and the anterior lens element may be fabricated as one single piece or two separate pieces. One or more holes are created in the posterior lens element, near the edges of the posterior lens element, for filling a cavity, described below, with optical fluid at step 230.

At step 220, the anterior lens element, the posterior lens element, and the main frame are assembled and sealed to form a cavity (e.g., cavity 106 (shown in FIGS. 1B and 1C)). The anterior lens element, the posterior lens element, and the main frame may be bonded together to make a seal in a peripheral non-optic portion of the lens body (e.g., lens body 102), by chemical bonding, thermal bonding, UV bonding or other appropriate types of bonding, with the cavity therebetween.

At step 230, the cavity is filled with an optical fluid, such as silicone oil through the one or more holes in the posterior lens element, by a lens manufacturing technique known to one of ordinary skill in the art. In certain embodiments, the cavity is filled using a syringe-assisted method. The one or more holes in the posterior lens element are subsequently sealed.

### System for Designing an IOL

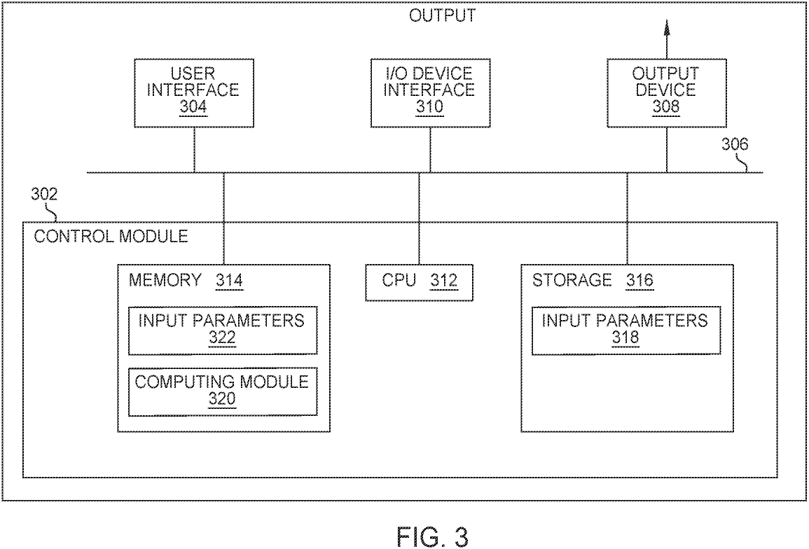

FIG. 3 depicts an exemplary system 300 for designing, configuring, and/or forming an IOL 100. As shown, the system 300 includes, without limitation, a control module 302, a user interface display 304, an interconnect 306, an output device 308, and at least one I/O device interface 310, which may allow for the connection of various I/O devices (e.g., keyboards, displays, mouse devices, pen input, etc.) to the system 300.

The control module 302 includes a central processing unit (CPU) 312, a memory 314, and a storage 316. The CPU 312 may retrieve and execute programming instructions stored in the memory 314. Similarly, the CPU 312 may retrieve and store application data residing in the memory 314. The interconnect 306 transmits programming instructions and application data, among CPU 312, the I/O device interface 310, the user interface display 304, the memory 314, the storage 316, output device 308, etc. The CPU 312 can represent a single CPU, multiple CPUs, a single CPU having multiple processing cores, and the like. Additionally, in certain embodiments, the memory 314 represents volatile memory, such as random access memory. Furthermore, in certain embodiments, the storage 316 may be non-volatile memory, such as a disk drive, solid state drive, or a collection of storage devices distributed across multiple storage systems.

As shown, the storage 316 includes input parameters 318. The input parameters 318 include a lens base power, and refractive indices and Abbe numbers of lens elements and an optical fluid to fill a cavity between the lens elements. The memory 314 includes a computing module 320 for computing control parameters, such as shapes of the lens elements (e.g., radii of curvatures and thickness). In addition, the memory 314 further includes input parameters 322.

In certain embodiments, input parameters 322 correspond to input parameters 318 or at least a subset thereof. In such embodiments, during the computation of the control parameters, the input parameters 322 are retrieved from the storage 316 and executed in the memory 314. In such an example, the computing module 320 comprises executable instructions for computing the control parameters, based on the input parameters 322. In certain other embodiments, input parameters 322 correspond to parameters received from a user through user interface display 304. In such embodiments, the computing module 320 comprises executable instructions for computing the control parameters, based on information received from the user interface display 304.

In certain embodiments, the computed control parameters, are output via the output device 308 to a lens manufacturing system that is configured to receive the control parameters and form a lens accordingly. In certain other embodiments, the system 300 itself is representative of at least a part of a lens manufacturing systems. In such embodiments, the control module 302 then causes hardware components (not shown) of system 300 to form the lens according to the control parameters by the operations 200 described above. The details of a lens manufacturing system are known to one of ordinary skill in the art and are omitted here for brevity.

### Method for Forming an IOL

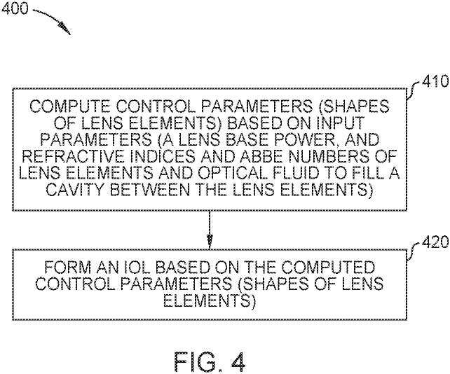

FIG. 4 depicts example operations 400 for forming an IOL (e.g., IOL 100). In some embodiments, the step 410 of operations 400 is performed by one system (e.g., the system 300) while step 420 is performed by a lens manufacturing system. In some other embodiments, both steps 410 and 420 are performed by a lens manufacturing system.

At step 410, control parameters, such as shapes of the lens elements 102A and 102P (e.g., radii of curvatures and thicknesses), are computed based on input parameters (e.g., a lens base power, and refractive indices and Abbe numbers of lens elements and optical fluid to fill a cavity between the lens elements). A variety of methods and techniques or algorithms may be used for selecting appropriate radii of curvatures and thicknesses for lens elements 102A and 102P in order to reduce chromatic aberrations. For example, a broad range of visual spectra may be used to calculate an optical power of each surface for a given dispersive material. Then appropriate radii of curvatures and thicknesses for lens elements 102A and 102P may be obtained using the calculated power and the optical properties of the dispersive material.

At step 420, an IOL (e.g., IOL 100) based on the computed control parameters, such as shapes of the lens elements 102A and 102P (e.g., radii of curvatures and thickness), is formed according to the operations 200 described above, using appropriate methods, systems, and devices typically used for manufacturing lenses, as known to one of ordinary skill in the art.

The embodiments described herein provide presbyopia-correcting IOLs in which chromatic aberration is corrected, while simultaneously avoiding the introduction of visual disturbances (e.g., halos, glare) more common to diffractive presbyopia-correcting IOLs. The presbyopia-correcting IOLs according to the embodiments described herein can be fabricated by producing an anterior lens element and a posterior lens element, bonding the anterior and posterior lens elements to form a cavity therebetween, and filling the cavity with an optical fluid. The methods described herein may simplify the conventional fabrication processes of such an IOL.

While the foregoing is directed to embodiments of the present disclosure, other and further embodiments of the disclosure may be devised without departing from the basic scope thereof, and the scope thereof is determined by the claims that follow.

1. An intraocular lens (IOL), comprising:
a lens body having an anterior lens element and a posterior lens element; and
an optical fluid in a cavity formed between the anterior lens element and the posterior lens element, wherein
the anterior lens element and the posterior lens element each comprise a lens material having a first Abbe number, and
the optical fluid has a second Abbe number that is less than the first Abbe number.

2. The IOL of claim 1, wherein the anterior lens element comprises refractive structures on an external surface of the anterior lens element.

3. The IOL of claim 1, wherein:
the first Abbe number is between 37 and 50, and
the second Abbe number is between 40 and 50.

4. The IOL of claim 1, wherein the lens material comprises hydrophobic acrylic polymeric material.

5. The IOL of claim 1, wherein the optical fluid comprises silicone oil.

6. The IOL of claim 1, further comprising a main frame coupled to the lens body, wherein the main frame comprises one or more haptics.

7. The IOL of claim 6, wherein the main frame is fabricated together as a single piece with the anterior lens element.

8. A method for fabricating an intraocular lens (IOL), comprising:
fabricating an anterior lens element and a posterior lens element of a lens body;
bonding the anterior lens element and the posterior lens element to form a cavity therebetweeen; and
filling the cavity with an optical fluid, wherein
the anterior lens element and the posterior lens element each comprise a lens material having a first Abbe number, and
the optical fluid has a second Abbe number that is less than the first Abbe number.

9. The method of claim 8, wherein the anterior lens element comprises refractive structures on an external surface of the anterior lens element.

10. The method of claim 8, wherein:
the first Abbe number is between 37 and 50, and
the second Abbe number is between 40 and 50.

11. The method of claim 8, wherein the lens material comprises hydrophobic acrylic polymeric material.

12. The method of claim 8, wherein the optical fluid comprises silicone oil.

13. The method of claim 8, further comprising bonding the lens body to a main frame.

14. The method of claim 13, wherein the main frame comprises one or more haptics.

15. A method for configuring an intraocular lens (IOL), comprising:
computing a radius of curvature of an anterior lens element of a lens body of an IOL and a radius of curvature of a posterior lens element of the lens body of the IOL, based on a lens base power of the IOL, a first refractive index and a first Abbe number of the anterior lens element and the posterior lens element, and a second refractive index and a second Abbe number of an optical fluid to fill a cavity between the anterior lens element and the posterior lens element; and
forming the IOL or causing the IOL to be formed based on the computed radii of curvature of the anterior lens element and the posterior lens element.

16. The method of claim 15, wherein the anterior lens element comprises refractive structures on an external surface of the anterior lens element.

17. The method of claim 15, wherein:
the first Abbe number is between 37 and 50, and
the second Abbe number is between 40 and 50.

18. The method of claim 15, wherein the anterior lens element and the posterior lens element comprise hydrophobic acrylic polymeric material.

19. The method of claim 15, wherein the optical fluid comprises silicone oil.

20. The method of claim 15, further comprising boding the lens body to a main frame, wherein the main frame comprises one or more haptics.
